(19) United States
(12) Patent Application Publication
(10) Pub. No.: US 2019/0380800 A1
(43) Pub. Date: Dec. 19, 2019

(54) MEDICAL TREATMENT TOOL

(71) Applicant: OLYMPUS CORPORATION, Tokyo (JP)

(72) Inventors: Shuya JOGASAKI, Tokyo (JP); Yoshiyuki KUMADA, Tokyo (JP); Kayuri KIMURA, Saitama (JP)

(73) Assignee: OLYMPUS CORPORATION, Tokyo (JP)

(21) Appl. No.: 16/556,514

(22) Filed: Aug. 30, 2019

Related U.S. Application Data
(63) Continuation of application No. PCT/JP2017/012908, filed on Mar. 29, 2017.

(51) Int. Cl. A61B 34/00; A61B 17/29

(52) U.S. Cl. CPC A61B 34/71; A61B 2017/2903; A61B 17/29

(57) ABSTRACT

A medical-treatment-tool according to the present invention is provided with: a inserting-portion; a gripping-piece pivots about a shaft orthogonal to a longitudinal axis of the inserting-portion; a driving-portion generates a motive power with which the gripping-piece is driven; a first motive-power transmitting member transmits the motive power to the inserting-portion; a motive-power amplifying mechanism amplifies the motive power; a pulley secured to the shaft of the gripping-piece; a wire-like second motive-power transmitting member wound around the pulley, transmits amplified the motive power to the pulley, and rotates the pulley about the shaft, thus causing the gripping-piece to be pivoted. The motive-power amplifying mechanism is a movable-pulley supported to move in a direction along the longitudinal axis of the inserting-portion, the first motive-power transmitting member is a wire-like member wound around the movable-pulley, and end of the second motive-power transmitting member is attached to the rotation shaft of the movable-pulley.

### CROSS-REFERENCE TO RELATED APPLICATIONS

This is a continuation of International Application PCT/JP2017/012908, with an international filing date of Mar. 29, 2017, which is hereby incorporated by reference herein in its entirety.

### TECHNICAL FIELD

The present invention relates to a medical treatment tool.

### BACKGROUND ART

There is a known medical treatment tool with which a procedure is performed by gripping biological tissue or the like (for example, see Japanese Unexamined Patent Application, Publication No. 2012-187311). With this medical treatment tool, a gripping portion disposed at a distal end of a long, thin inserting portion is closed by means of a pulling force applied to an operating portion disposed at a proximal end of the inserting portion, and a high gripping force is achieved in the gripping portion by amplifying the pulling force by means of a toggle mechanism.

### SUMMARY OF INVENTION

An aspect of the present invention is a medical treatment tool including: a long, thin, flexible inserting portion; a gripping piece that is supported at a distal end of the inserting portion so as to be pivoted about a shaft that is orthogonal to a longitudinal axis of the inserting portion; a driving portion that is disposed at a proximal end of the inserting portion and that generates a motive power with which the gripping piece is driven; a first motive-power transmitting member that transmits the motive power generated by the driving portion to the distal end of the inserting portion; a motive-power amplifying mechanism that amplifies the motive power transmitted by the first motive-power transmitting member; a pulley secured to the shaft of the gripping piece; a wire-like second motive-power transmitting member that is wound around the pulley, that transmits the motive power amplified by the motive-power amplifying mechanism to the pulley, and that rotates the pulley about the shaft, thus causing the gripping piece to be pivoted, wherein the motive-power amplifying mechanism is a movable pulley that is supported so as to move in a direction along the longitudinal axis of the inserting portion, the first motive-power transmitting member is a wire-like member that is wound around the movable pulley, and one end of the second motive-power transmitting member is attached to the rotation shaft of the movable pulley.

### BRIEF DESCRIPTION OF DRAWINGS

FIG. 1 is a front view schematically showing a state in which gripping pieces of a medical treatment tool according to a first embodiment of the present invention are closed.

FIG. 2 is a front view schematically showing a state in which the gripping pieces of the medical treatment tool in FIG. 1 are open.

FIG. 3 is a front view schematically showing a state in which gripping pieces of a medical treatment tool according to a second embodiment of the present invention are closed.

FIG. 4 is a front view schematically showing a state in which the gripping pieces of the medical treatment tool in FIG. 3 are open.

FIG. 5 is a front view schematically showing a state in which gripping pieces of a first modification of the medical treatment tool in FIG. 3 are closed.

FIG. 6 is a front view schematically showing a state in which the gripping pieces of the medical treatment tool in FIG. 5 are open.

FIG. 7 is a front view schematically showing a state in which gripping pieces of a second modification of the medical treatment tool in FIG. 3 are closed.

FIG. 8 is a front view schematically showing a state in which the gripping pieces of the medical treatment tool in FIG. 7 are open.

FIG. 9 is a front view schematically showing a state in which gripping pieces of a third modification of the medical treatment tool in FIG. 3 are closed.

FIG. 10 is a front view for explaining forces that act when opening one of the gripping pieces of the medical treatment tool in FIG. 9.

FIG. 11 is a front view for explaining forces that act when opening the other gripping piece of the medical treatment tool in FIG. 9.

FIG. 12 is a front view for explaining a fourth modification of the medical treatment tool in FIG. 3 in the case in which a flexing joint is provided.

FIG. 13 is a front view schematically showing a state in which gripping pieces of a medical treatment tool according to a third embodiment of the present invention are closed.

FIG. 14 is a front view schematically showing a state in which the gripping pieces of the medical treatment tool in FIG. 13 are open.

### DESCRIPTION OF EMBODIMENTS

A medical treatment tool 1 according to a first embodiment of the present invention will be described below with reference to the drawings.

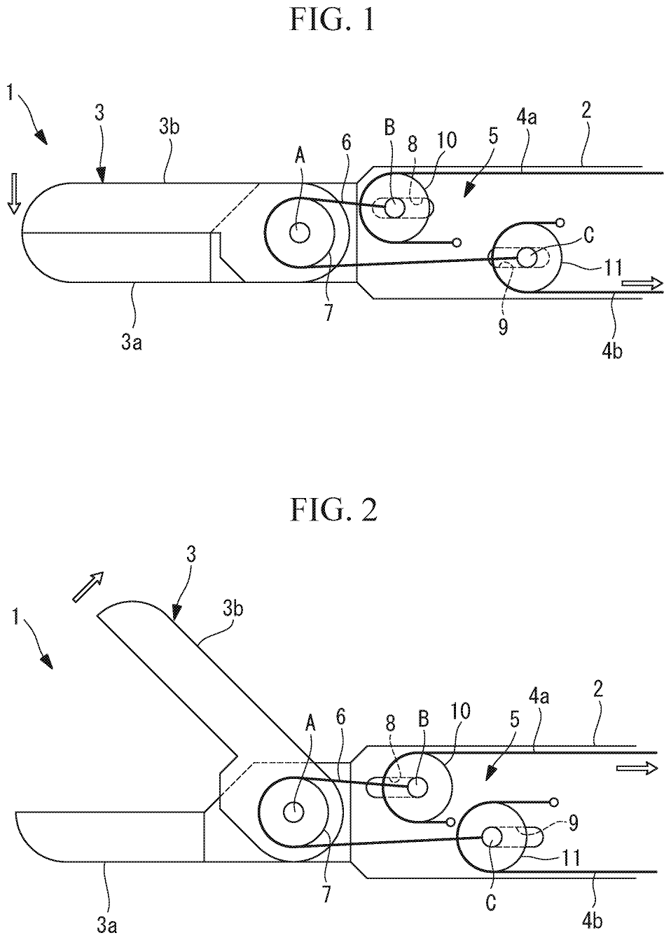

As shown in FIGS. 1 and 2, the medical treatment tool 1 according to this embodiment is provided with: a long, thin, flexible inserting portion 2; a gripping portion 3 provided at a distal end of the inserting portion 2; a driving portion (not shown) provided at a proximal end of the inserting portion 2; first wires (first motive-power transmitting members, wire-like members) 4a and 4b that transmit pulling forces (motive powers) generated by the driving portion; an amplifying mechanism (motive-power amplifying mechanism) 5 that amplifies the pulling forces; a second wire (second motive-power transmitting member) 6 that transmits the forces amplified by the amplifying mechanism 5; and a pulley 7 that converts the pulling forces in the second wire 6 to a force for opening/closing the gripping portion 3.

The gripping portion 3 is provided with: a stationary gripping piece (gripping piece) 3a that is secured to the distal end of the inserting portion 2; and a movable gripping piece (gripping piece) 3b that is supported so as to be pivotable about a shaft A that extends in a direction orthogonal to the longitudinal axis of the inserting portion 2. The pulley 7 is secured to the shaft A of the movable gripping piece 3b, is rotated, as a result of the second wire 6 wound around the pulley 7 being pulled, in the direction of a moment generated by the pulling, and causes the movable gripping piece 3b to which the pulley 7 is secured to be pivoted.

As a result of being pivoted in one direction, the movable gripping piece 3b is pivoted in the direction in which a gap between the stationary gripping piece 3a and the movable gripping piece 3b is decreased, and thus, it is possible to grip a treatment target site between the stationary gripping piece 3a and the movable gripping piece 3b. As a result of being pivoted in the other direction, the movable gripping piece 3b is pivoted in the direction in which the gap between the stationary gripping piece 3a and the movable gripping piece 3b is increased, and thus, it is possible to widen the treatment target site.

The driving portion is a portion that electrically or manually generates pulling forces, and applies the generated pulling forces to proximal ends of the first wires 4a and 4b.

The amplifying mechanism 5 is provided with: two elongated holes 8 and 9 that are provided in the inserting portion 2 along the longitudinal-axis direction of the inserting portion 2; and two movable pulleys 10 and 11 that are accommodated in the individual elongated holes 8 and 9 so as to be rotatable about rotation shafts B and C and movable in the longitudinal-axis direction.

The first wires 4a and 4b are separately provided in the movable pulleys 10 and 11, proximal ends thereof are connected to the driving portion, and distal ends thereof are secured to the inserting portion 2 after being turned back by approximately 180° by being wound around the movable pulleys 10 and 11.

The second wire 6 is wound around the pulley 7 and both ends thereof are individually attached to the rotation shafts B and C of the two movable pulleys 10 and 11, and the second wire 6 is secured to the pulley 7 at an intermediate position in the length direction.

By doing so, as a result of the movable pulley 11 being moved toward the proximal end along the longitudinal axis, the second wire 6 attached to the rotation shaft C of the movable pulley 11 is pulled, and thus, it is possible to rotate the pulley 11 in one direction in accordance with the generated moment. As a result of the pulley 10 being moved toward the proximal end along the longitudinal axis, the second wire 6 attached to the rotation shaft B of the movable pulley 10 is pulled, and thus, it is possible to rotate the pulley 10 in the other direction in accordance with the generated moment.

The operation of the medical treatment tool 1 according to this embodiment, thus configured, will be described below.

In order to treat a treatment target site by using the medical treatment tool 1 according to this embodiment, the inserting portion 2 is inserted into a body from the distal-end side of the inserting portion 2, and the gripping portion 3 is brought close to the treatment target site in a state in which the distal end of the gripping portion 3 is open.

Then, the driving portion is activated to generate the pulling force with which the first wire 4a or 4b is pulled toward the proximal end. The pulling force applied to the first wire 4a is transmitted to the distal end of the inserting portion 2 by the first wire 4a and pulls the movable pulley 10, around which the first wire 4a is wound, toward the proximal end, and the pulling force applied to the first wire 4b is transmitted to the distal end of the inserting portion 2 by the first wire 4b and pulls the movable pulley 11, around which the first wire 4b is wound, toward the proximal end. Because the first wires 4a and 4b wound around the movable pulleys 10 and 11 extend toward the proximal end substantially parallel to each other on either side of the rotation shafts B and C of the movable pulleys 10 and 11, the pulling forces acting on the two first wires 4a and 4b act on the rotation shafts B and C of the movable pulleys 10 and 11, and thus, the rotation shafts B and C are pulled toward the proximal end by forces that are twice the pulling force.

Specifically, the movable pulleys 10 and 11 serve as moving pulleys, the movable pulleys 10 and 11 are moved by an amount that is reduced by half with respect to the amount of movement due to the pulling by the first wires 4a and 4b, and, consequently, a force that is doubled with respect to the pulling force in the first wires 4a and 4b acts on the second wire 6 attached to the rotation shafts B and C. By doing so, the pulley 7 around which the second wire 6 is wound is rotated by the large force amplified by the movable pulleys 10 and 11, and thus, the movable gripping piece 3b is pivoted.

As shown in FIG. 1, as a result of applying the pulling force to the first wire 4b, the pulling force amplified by the movable pulley 11 causes, via the second wire 6, the pulley 7 to be rotated in one direction, and thus, it is possible to pivot the movable gripping piece 3b in the closing direction with respect to the stationary gripping piece 3a. By doing so, it is possible to grip the treatment target site with a large force.

As shown in FIG. 2, as a result of applying the pulling force to the first wire 4a, the pulling force amplified by the movable pulley 10 causes, via the second wire 6, the pulley 7 to be rotated in the opposite direction, and thus, it is possible to pivot the movable gripping piece 3b in the opening direction with respect to the stationary gripping piece 3a. By doing so, it is possible to widen the treatment target site with a large force.

As has been described above, with the medical treatment tool 1 according to this embodiment, because the movable gripping piece 3b is pivoted by amplifying the pulling force applied to the first wires 4a and 4b by means of the amplifying mechanism 5, it is possible to treat the treatment target site with a large force. In this case, because the amplifying mechanism 5 is constituted by the movable pulleys 10 and 11 that are moved in the longitudinal-axis direction of the inserting portion 2, the amplifying mechanism 5 does not expand in a radial direction, as in a toggle mechanism, in the operation of pivoting the movable gripping piece 3b. As a result, there is an advantage in that it is possible to reduce the diameter of the inserting portion 2.

In particular, by utilizing the movable pulleys 10 and 11 that serve as moving pulleys, it is possible to amplify, with a simple configuration, the pulling forces applied to the first wires 4a and 4b that extend in the longitudinal direction of the inserting portion 2 and to generate large pulling forces that act in the longitudinal direction.

In this embodiment, because the movable pulleys 10 and 11 that constitute the amplifying mechanism 5 are disposed individually at the two ends of the second wire 6 wound around the pulley 7, there is an advantage in that it is possible to perform treatment with a large force by amplifying the pulling force both in the operation to open the movable gripping piece 3b and the operation to close the movable gripping piece 3b.

Next, a medical treatment tool 12 according to a second embodiment of the present invention will be described below with reference to the drawings.

In describing this embodiment, portions having the same configurations as those of the medical treatment tool 1 according to the first embodiment, described above, will be given the same reference signs and descriptions thereof will be omitted.

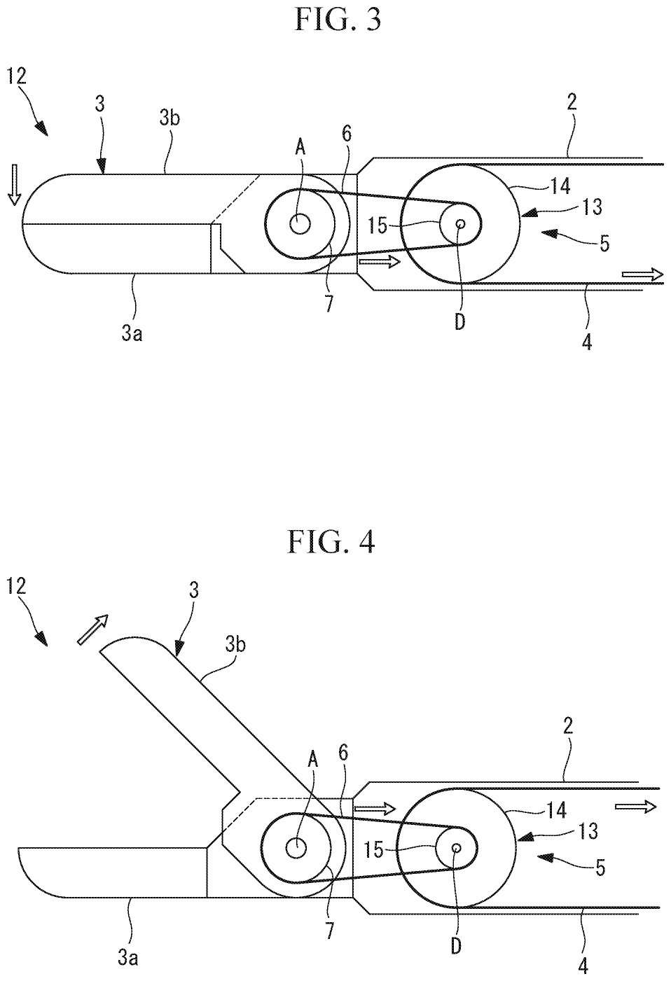

As shown in FIGS. 3 and 4, the medical treatment tool 12 according to this embodiment is provided with a stepped pulley (motive-power amplifying mechanism) 13 instead of the movable pulleys 10 and 11 in the medical treatment tool 1 according to the first embodiment.

The stepped pulley 13 is attached to the inserting portion 2 so as to be rotatable about a rotation shaft D that is orthogonal to the longitudinal axis of the inserting portion 2, and is coaxially provided with a large-diameter portion 14 and a small-diameter portion 15 that has a smaller diameter than the large-diameter portion 14.

An intermediate position of a first wire 4 in the length direction is secured to the large-diameter portion 14 of the stepped pulley 13, and two ends of the first wire 4 extend toward the proximal end of the inserting portion 2. By doing so, it is possible to rotate the stepped pulley 13 in one direction by pulling one end of the first wire 4, and to rotate the stepped pulley 13 in the other direction by pulling the other end of the first wire 4.

The second wire 6 is formed in a loop shape, and bridges across the pulley 7 secured to the movable gripping piece 3b and the small-diameter portion 15 of the stepped pulley 13. Intermediate positions of the second wire 6 in the length direction are secured to the pulley 7 and the small-diameter portion 15.

With the medical treatment tool 12 according to this embodiment, thus configured, as shown in FIG. 3, as a result of applying a pulling force to one end of the first wire 4 by means of the driving portion, the stepped pulley 13 is rotated in one direction, and the pulling force acts on the second wire 6 secured to the small-diameter portion 15. Because the small-diameter portion 15 and the large-diameter portion 14 have diameters of a predetermined ratio, the pulling force applied to the first wire 4 acts on the second wire 6 after being amplified by an amount corresponding to the ratio between the small-diameter portion 15 and the large-diameter portion 14.

As shown in FIG. 4, as a result of applying the pulling force to the other end of the first wire 4 by means of the driving portion, the stepped pulley 13 is rotated to the other direction, the pulling force applied to the first wire 4 acts on the second wire 6 secured to the small-diameter portion 15 in the form of the pulling force that is amplified by an amount corresponding to the ratio between the small-diameter portion 15 and the large-diameter portion 14.

By doing so, it is possible to pull the second wire 6 with a force that is greater than the pulling force generated by the driving portion and to treat the treatment target site with a large force.

In this case also, because the stepped pulley 13 constituting the amplifying mechanism 5 is secured to the inserting portion 2, the size of the inserting portion 2 does not change in a radial direction in the pivoting operation of the movable gripping piece 3b, and thus, there is an advantage in that it is possible to reduce the diameter of the inserting portion 2.

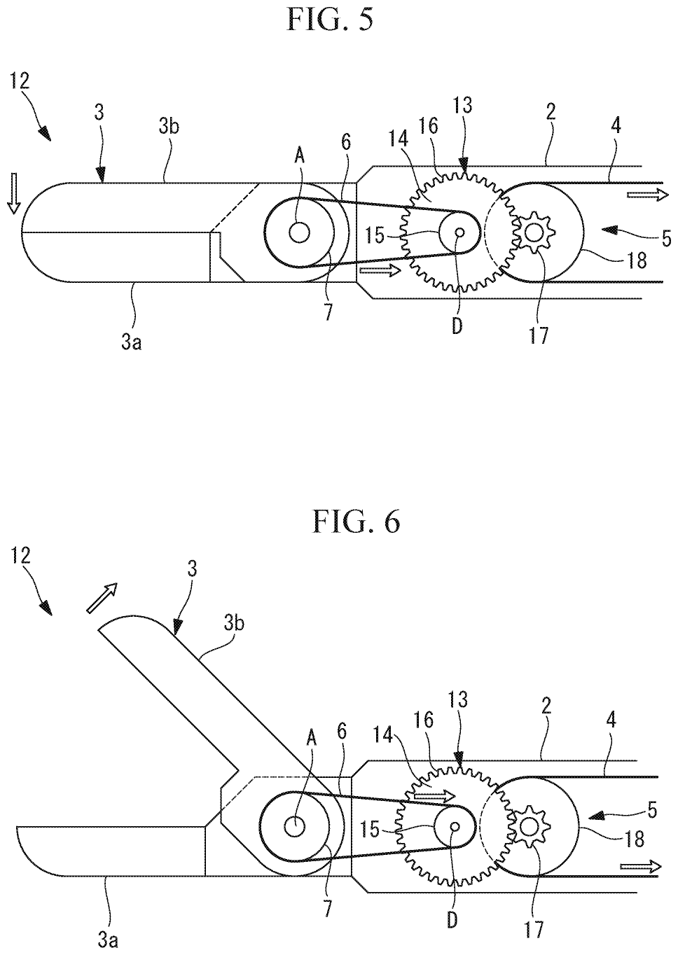

In this embodiment, the pulling force is applied to the large-diameter portion 14 by means of the stepped pulley 13, and the pulling force that acts on the second wire 6 wound around the small-diameter portion 15 is amplified; however, alternatively, as shown in FIGS. 5 and 6, a driven gear 16 may be provided in the large-diameter portion 14, a driving gear 17 that engages with the driven gear 16 and a pulley 18 that is secured so as to be coaxial with the driving gear 17 may be provided, and the pulley 18 may be rotated by means of the first wire 4. In this case, it is preferable that the diameter of the pulley 18 be set so as to be greater than the diameter of a pitch circle of the driving gear 17.

By doing so, the pulling force applied to the first wire 4 is applied to the driven gear 16 after being amplified by an amount corresponding to a ratio between the diameter of the pulley 18 and the diameter of the pitch circle of the driving gear 17, and is applied to the second wire 6 after being amplified by an amount corresponding to a ratio between the diameter of the pitch circle of the driven gear 16 and the diameter of the small-diameter portion 15. Specifically, because the pulling force applied to the first wire 4 is applied to the second wire 6 after being amplified in two stages, there is an advantage in that it is possible to treat the treatment target site with a greater force.

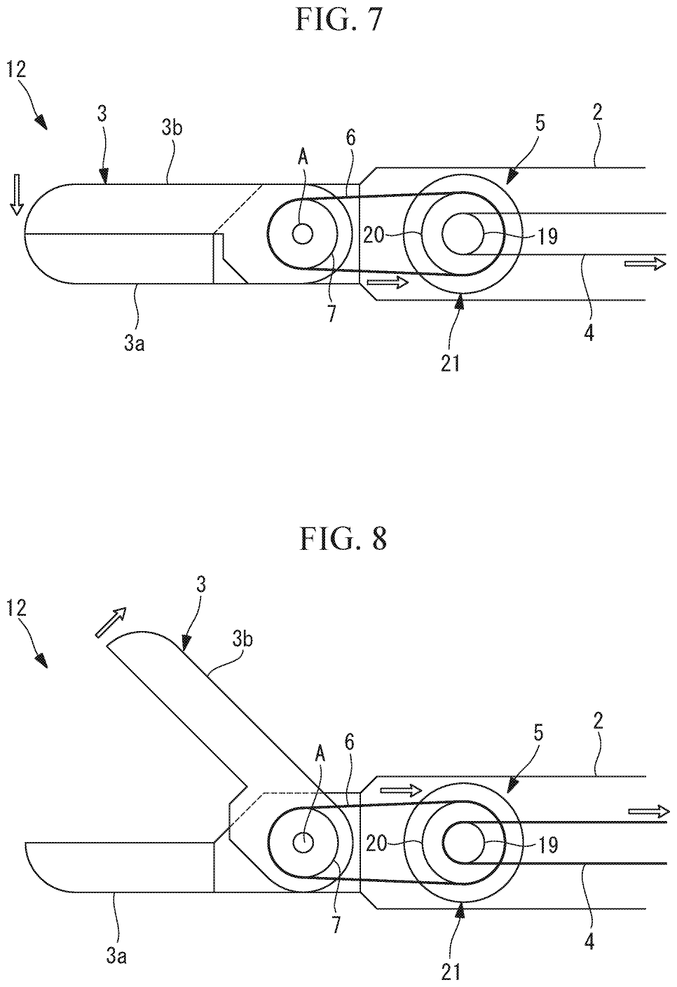

As shown in FIGS. 7 and 8, instead of the stepped pulley 13, a decelerator 21 that has a planetary gear (not shown) in the interior thereof, and that rotates an output shaft 20 by decelerating the rotational speed of an input shaft 19 by a predetermined deceleration ratio may be employed.

In the example shown in FIGS. 7 and 8, the first wire 4 is wound around the input shaft 19, and the second wire 6 is wound around the output shaft 20. By doing so, because the pulling force applied to the first wire 4 acts on the second wire 6 after being amplified by an amount corresponding to the deceleration ratio of the decelerator 21, there is an advantage in that it is possible to treat the treatment target site with a greater force.

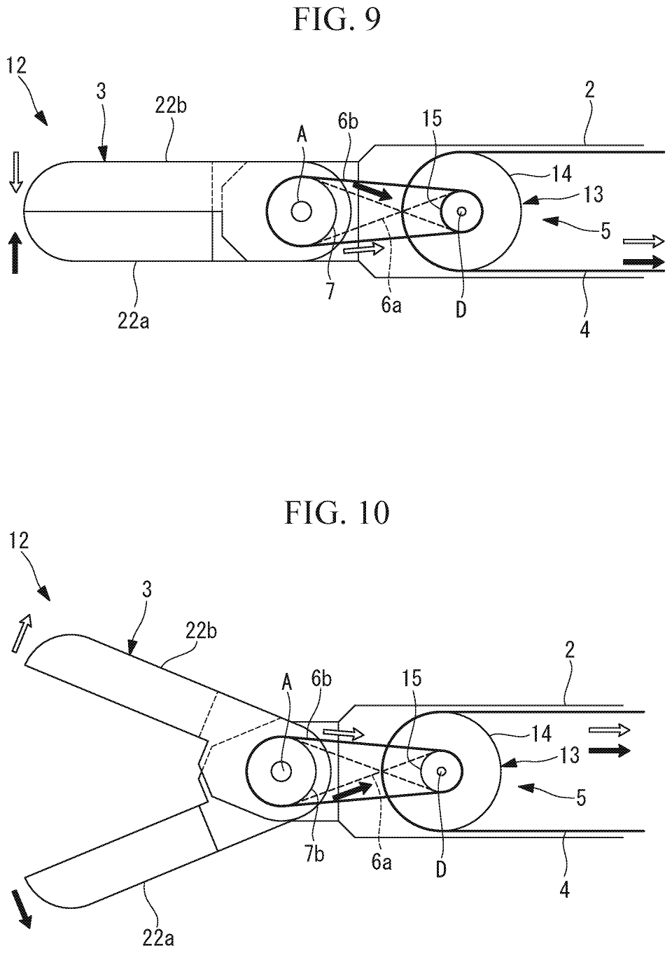

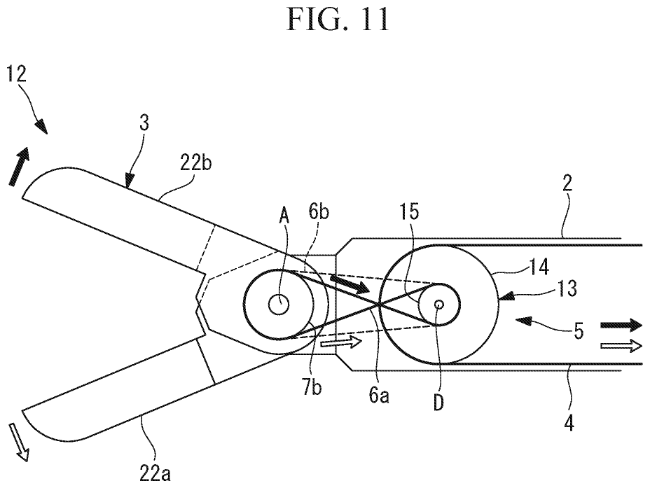

Although this embodiment has been described in terms of the case in which the stationary gripping piece 3a is fixed and only the movable gripping piece 3b is pivoted, alternatively, as shown in FIGS. 9 to 11, a pair of gripping pieces 22a and 22b may be pivoted. In this case, separate pulleys 7a and 7b may be secured to the individual gripping pieces 22a and 22b, and, as shown in FIG. 11, a loop-shaped second wire 6a to be wound around the pulley 7a of the gripping piece 22a may be wound around the small-diameter portion 15 after being crossed into an 8-shape.

By doing so, as shown in FIG. 9, as a result of applying a pulling force to one end of the first wire 4, the stepped pulley 13 is rotated in one direction, and the second wire 6a causes the gripping piece 22a to be pivoted in the closing direction, and a second wire 6b also causes the gripping piece 22b to be pivoted in the closing direction; therefore, it is possible to close the pair of gripping pieces 22a and 22b with a large force by pivoting the gripping pieces 22a and 22b in a synchronized manner.

As shown in FIGS. 10 and 11, as a result of applying a pulling force to the other end of the first wire 4, the stepped pulley 13 is rotated in the other direction, the second wire 6a causes the gripping piece 22a to be pivoted in the opening direction, and the second wire 6b also causes the gripping piece 22b to be pivoted in the opening direction; therefore, it is possible to open the pair of gripping pieces 22a and 22b with a large force by pivoting the gripping pieces 22a and 22b in a synchronized manner.

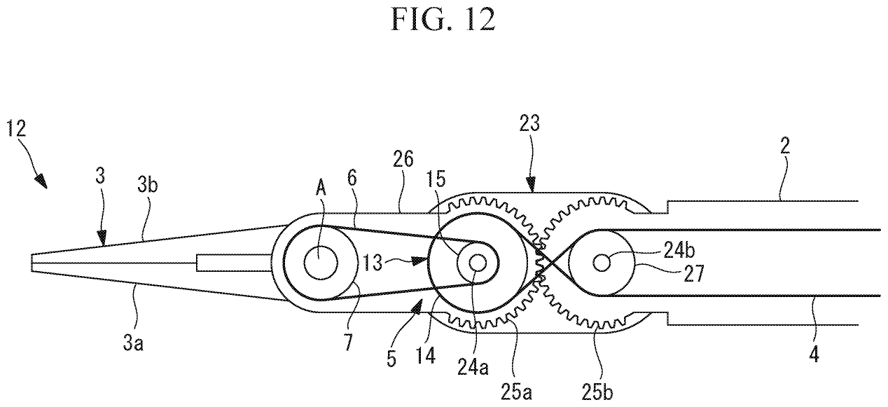

In this embodiment, as shown in FIG. 12, a flexing joint 23 with which it is possible to change the direction of the gripping portion 3 about a shaft that is orthogonal to the longitudinal axis of the inserting portion 2 may be provided on the proximal-end side of the gripping portion 3.

The flexing joint 23 may be configured such that, for example, gears 25a and 25b that engage with each other are provided about two parallel shafts 24a and 24b, respectively, and, as a result of applying a moment, by means of an operating wire (not shown), to a member 26 in which the gear 25a on the distal-end side is provided, the gripping portion 3 is pivoted while keeping the gear 25a in rolling contact about the shaft 24b of the gear 25b on the proximal-end side gear 25b.

Also, in this case, even if the flexing joint 23 is flexed by providing, of the two shafts 24a and 24b that constitute the flexing joint 23, the stepped pulley 13 on the shaft 24a on the distal-end side and an idler pulley 27 on the shaft 24b on the proximal-end side, and by winding the first wire 4 being crossed between the large-diameter portion 14 of the stepped pulley 13 and the idler pulley 27, as shown in FIG. 12, it is possible to rotationally drive the stepped pulley 13 by means of the pulling force in the first wire 4.

Alternatively, the stepped pulley 13 may be provided on the shaft 24b on the proximal-end side, the idler pulley 27 may be provided on the shaft 24a on the distal-end side, and the second wire 6 may be wound by being crossed between the small-diameter portion 15 of the stepped pulley 13 and the idler pulley 27.

Next, a medical treatment tool 28 according to a third embodiment of the present invention will be described below with reference to the drawings.

In describing this embodiment, portions having the same configurations as those of the medical treatment tool 12 according to the second embodiment, described above, will be given the same reference signs and descriptions thereof will be omitted.

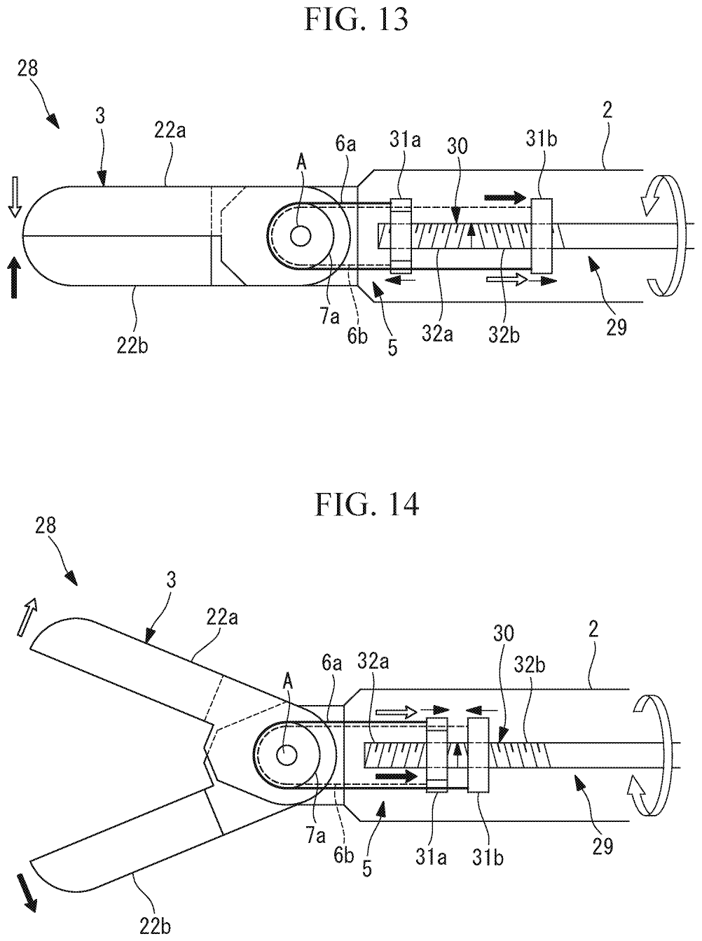

As shown in FIGS. 13 and 14, the medical treatment tool 28 according to this embodiment employs a shaft (first motive-power transmitting member) 29 instead of the first wire 4 in the medical treatment tool 12 according to the second embodiment, and is provided with, instead of the stepped pulley 13, movable bodies 31a and 31b that are provided at a distal end of the shaft 29 and that have a feed screw 30 and a screw hole to which the feed screw 30 is fastened, so as to serve as the amplifying mechanism 5.

The feed screw 30 is provided with two types of feed screw portions 32a and 32b that are next to each other in the longitudinal-axis direction at the distal end of the shaft 29 and that have a relationship in which the threading directions thereof are opposite from each other. The movable bodies 31a and 31b are individually fastened on these two types of feed screw portions 32a and 32b. By doing so, as a result of rotating the shaft 29 in one direction about the longitudinal axis, the two movable bodies 31a and 31b are moved in directions in which the movable bodies 31a and 31b separate from each other along the longitudinal-axis direction, and, as a result of rotating the shaft 29 in the other direction about the longitudinal axis, the movable bodies 31a and 31b are moved in directions in which the movable bodies 31a and 31b are brought close to each other along the longitudinal-axis direction.

One end of the second wire 6 wound around the pulley 7a, which is secured to the gripping piece 22a, is secured to the movable body 31a, and the other end thereof is secured to the movable body 31b. With the second wire 6b wound around the pulley 7b secured to the gripping piece 22b also, one end thereof is secured to the movable body 31a and the other end thereof is secured to the movable body 31b.

By doing so, as shown in FIG. 13, when the movable bodies 31a and 31b are moved in the directions in which the movable bodies 31a and 31b are separated from each other as a result of the shaft 29 being rotated in one direction, the pulleys 7a and 7b, around which the two second wires 6a and 6b are individually wound, are rotated so as to pivot the gripping pieces 22a and 22b, to which the pulleys 7a and 7b are secured, in directions in which the gripping pieces 22a and 22b close with respect to each other. As shown in FIG. 14, when the movable bodies 31a and 31b are moved in the directions in which the movable bodies 31a and 31b are brought close to each other as a result of the shaft 29 being rotated in the other direction, the pulleys 7a and 7b, around which the two second wires 6a and 6b are individually wound, are rotated so as to pivot the gripping pieces 22a and 22b, to which the pulleys 7a and 7b are secured, in directions in which the gripping pieces 22a and 22b open with respect to each other.

In this case, by setting the lead of the feed screw 30 to be sufficiently small, it is possible to sufficiently amplify the pulling forces that act on the second wires 6a and 6b with respect to the rotational force applied to the shaft 29, and thus, there is an advantage in that it is possible to treat the treatment target site with a large force. Also, because the feed screw 30 and the movable bodies 31a and 31b are employed so as to serve as the amplifying mechanism 5, the amplifying mechanism 5 is not displaced in a radial direction in the opening/closing operations of the gripping pieces 22a and 22b, and thus, there is an advantage in that it is possible to reduce the diameter of the inserting portion 2.

In this embodiment, the two feed screw portions 32a and 32b having a relationship in which the threading directions thereof are opposite from each other are provided in the shaft 29, and the two gripping pieces 22a and 22b are pivoted by means of a single shaft 29 in a synchronized manner; however, alternatively, the two gripping pieces 22a and 22b may be driven by means of separate shafts.

Although the individual embodiments, described above, are configured so that it is possible to apply a large force not only in the direction in which the gripping pieces 3a, 3b, 22a, and 22b close but also in the direction in which the gripping pieces 3a, 3b, 22a, and 22b open, it is permissible to amplify the motive power only in the closing direction or the opening direction and not in the other direction. Alternatively, the motive power may be amplified by using different amplification factors. Alternatively, regarding the other direction, the gripping pieces may constantly be biased by a spring (not shown) in the opening direction or the closing direction.

Each of the amplifying mechanisms 5 may be provided with multiple stages in series. By doing so, it is possible to further increase the amplification factor.

As a result, the following aspect is read from the above described embodiment of the present invention.

An aspect of the present invention is a medical treatment tool including: a long, thin, flexible inserting portion; a gripping piece that is supported at a distal end of the inserting portion so as to be pivoted about a shaft that is orthogonal to a longitudinal axis of the inserting portion; a driving portion that is disposed at a proximal end of the inserting portion and that generates a motive power with which the gripping piece is driven; a first motive-power transmitting member that transmits the motive power generated by the driving portion to the distal end of the inserting portion; a motive-power amplifying mechanism that amplifies the motive power transmitted by the first motive-power transmitting member; a pulley secured to the shaft of the gripping piece; a wire-like second motive-power transmitting member that is wound around the pulley, that transmits the motive power amplified by the motive-power amplifying mechanism to the pulley, and that rotates the pulley about the shaft, thus causing the gripping piece to be pivoted.

With this aspect, by inserting the flexible inserting portion into the body and by activating the driving portion disposed at the proximal end of the inserting portion, it is possible to perform treatment by pivoting the gripping piece at the distal end of the inserting portion. The motive power generated by the driving portion is transmitted to the distal end of the inserting portion by the first motive-power transmitting member and is transmitted to the pulley by the second motive-power transmitting member after being amplified by the motive-power amplifying mechanism, and the gripping piece is pivoted by means of the rotation of the pulley. Because the motive power generated by the driving portion is amplified by the motive-power amplifying mechanism, it is possible to operate the gripping piece with a low motive power, it is possible to convert the motive power that is transmitted in a small space by the pulley via the wire-like second motive-power transmitting member to the force that causes the gripping piece to be pivoted, and thus, it is possible to reduce the diameter of the inserting portion.

In the above-described aspect, the motive-power amplifying mechanism may be a movable pulley that is supported so as to be movable in a direction along the longitudinal axis of the inserting portion, the first motive-power transmitting member may be a wire-like member that is wound around the movable pulley, and one end of the second motive-power transmitting member may be attached to a rotation shaft of the movable pulley.

By doing so, as a result of pulling the first motive-power transmitting member, the movable pulley around which the first motive-power transmitting member is wound is moved in the direction along the longitudinal axis of the inserting portion, the second motive-power transmitting member attached to the rotation shaft of the movable pulley is pulled, the pulley around which the second motive-power transmitting member is wound is rotated, and thus, the gripping piece is pivoted. As a result of using the movable pulley as a moving pulley, because the force with which the first motive-power transmitting member is pulled is doubled and the second motive-power transmitting member is pulled with this force, it is possible to cause the gripping piece to be pivoted by amplifying the motive power generated by the driving portion. In this case, because the movable pulley is moved in the direction along the longitudinal axis of the inserting portion, it is possible to avoid an increase in the diameter of the inserting portion and it is possible to reduce the diameter thereof.

In the above-described aspect, the motive-power amplifying mechanism may be provided with a stepped pulley provided with a large-diameter portion and a small-diameter portion, the first motive-power transmitting member may be wound around the large-diameter portion, and the second motive-power transmitting member may be wound around the small-diameter portion.

By doing so, as a result of pulling the first motive-power transmitting member, the stepped pulley in which the first motive-power transmitting member is wound around the large-diameter portion is rotated, the second motive-power transmitting member wound around the small-diameter portion of the stepped pulley is pulled, the pulley is rotated, and thus, the gripping piece is pivoted. Regarding the force with which the second motive-power transmitting member is pulled, because the force with which the first motive-power transmitting member is pulled is amplified by an amount corresponding to the ratio between the large-diameter portion and the small-diameter portion, it is possible to treat the treatment target site with a large force. In this case also, as a result of keeping the outer diameter of the stepped pulley to a size that is equal to or less than the outer diameter of the inserting portion, it is possible to avoid an increase in the diameter of the inserting portion and it is possible to reduce the diameter thereof.

In the above-described aspect, the first motive-power transmitting member may be a shaft, the motive-power amplifying mechanism may be provided with a feed screw formed at a distal end of the shaft and a movable body having a screw hole to which the feed screw is fastened, and one end of the second motive-power transmitting member may be attached to the movable body.

By doing so, as a result of generating, by means of the driving portion, a driving force for rotating the shaft constituting the first motive-power transmitting member about the longitudinal axis, the rotation of the feed screw formed at the distal end of the shaft is converted to a linear movement of the movable body in the longitudinal-axis direction in accordance with the lead of the feed screw, the second motive-power transmitting member secured to the movable body is pulled in the longitudinal-axis direction, the pulley is rotated, and thus, it is possible to cause the gripping piece to be pivoted.

By setting the lead of the feed screw to be sufficiently small, it is possible to transmit the motive power applied to the first motive-power transmitting member to the second motive-power transmitting member after amplifying the motive power, and it is possible to treat the treatment target site with a large force. In this case also, as a result of keeping the outer diameter of the movable body to a size that is equal to or less than the outer diameter of the inserting portion, it is possible to avoid an increase in the diameter of the inserting portion and it is possible to reduce the diameter thereof.

In the above-described aspect, the motive-power amplifying mechanism may amplify motive powers for causing the gripping piece to be pivoted in both directions about the shaft.

By doing so, it is possible to execute both the operation in which the treatment target site is gripped with a large gripping force by closing the gripping piece and the operation in which the treatment target site is widen with a large force by opening the gripping piece.

In the above-described aspect, the motive-power amplifying mechanism may be provided with multiple stages in series.

By doing so, it is possible to amplify the motive power with a large amplification factor depending on the number of stages in the motive-power amplifying mechanism disposed in series.

In the above-described aspect, a flexing joint that flexes about a shaft that is parallel to the shaft of the gripping piece may be provided farther on the proximal-end side than the gripping piece is, and the stepped pulley may be attached so as to be rotatable about the shaft of the flexing joint.

By doing so, as a result of bending one of the first motive-power transmitting member that drives the stepped pulley and the second motive-power transmitting member that is pulled by the stepped pulley in accordance with flexing of the flexing joint, it is possible to treat the treatment target site with a large force by transmitting the motive power to the distal-end side beyond the flexing joint from the proximal-end side of the flexing joint.

### REFERENCE SIGNS LIST

1, 12, 28 medical treatment tool
2 inserting portion
3a stationary gripping piece (gripping piece)
3b movable gripping piece (gripping piece)
4, 4a, 4b first wire (first motive-power transmitting member, wire-like member)
5 amplifying mechanism (motive-power amplifying mechanism)
6, 6a, 6b second wire (second motive-power transmitting member)
7,7a,7b pulley
10, 11 movable pulley (motive-power amplifying mechanism)
13 stepped pulley (motive-power amplifying mechanism)
14 large-diameter portion
15 small-diameter portion
22a, 22b gripping piece
23 flexing joint
29 shaft (first motive-power transmitting member)
30 feed screw
31a, 31b movable body
A shaft
B, C rotation shaft

1. A medical treatment tool comprising:
a long, thin, flexible inserting portion;
a gripping piece that is supported at a distal end of the inserting portion so as to be pivoted about a shaft that is orthogonal to a longitudinal axis of the inserting portion;
a driving portion that is disposed at a proximal end of the inserting portion and that is configured to generate a motive power with which the gripping piece is driven;
a first motive-power transmitting member that is configured to transmit the motive power generated by the driving portion to the distal end of the inserting portion;
a motive-power amplifying mechanism that is configured to amplify the motive power transmitted by the first motive-power transmitting member;
a pulley secured to the shaft of the gripping piece;
a wire-like second motive-power transmitting member that is wound around the pulley, that is configured to transmit the motive power amplified by the motive-power amplifying mechanism to the pulley, and to rotate the pulley about the shaft, thus causing the gripping piece to be pivoted,
wherein the motive-power amplifying mechanism is a movable pulley that is supported so as to move in a direction along the longitudinal axis of the inserting portion,
the first motive-power transmitting member is a wire-like member that is wound around the movable pulley, and
one end of the second motive-power transmitting member is attached to the rotation shaft of the movable pulley.

2. A medical treatment tool according to claim 1, wherein the motive-power amplifying mechanism amplifies a motive power that causes the gripping piece to be pivoted in two directions about the shaft.

3. A medical treatment tool according to claim 1, wherein the motive-power amplifying mechanism is provided in multiple stages in series.
